(19) United States
(12) Patent Application Publication
(10) Pub. No.: US 2001/0042341 A1
(43) Pub. Date: Nov. 22, 2001

(54) Vase-added compositions for controlling plant and flower moisture transpiration rates

(76) Inventors: Mark William Hamersky, Hamilton, OH (US); Steven Daryl Smith, Fairfield, OH (US)

Correspondence Address: Richard S. Echler, The Procter & Gamble Company, Box 538707, Cincinnati, OH, 45253-8707, US

(21) Appl. No.: 09/760,037

(22) Filed: Jan. 12, 2001

Related U.S. Application Data
(60) Provisional application No. 60/176,090, filed on Jan. 14, 2000.

(51) Int. Cl. A01G 1/00; A01C 1/00; A01H 3/00; A01B 79/00; A01B 79/02; C07C 211/00; C07C 213/00; C07C 215/00; C07C 217/00; C07C 221/00

(52) U.S. Cl. 47/58.1; 564/281

(57) ABSTRACT

The present invention relates to compositions for controlling plant and flower moisture transpiration and thereby extending the period of time in which cut flowers can be displayed before senescence produces a flower which has exceeded its aesthetic value. The compositions of the present invention comprise:

a) from about 0.1% by weight, of a source of energy;

b) from about 5 ppm by weight, of one or more antimicrobials;

c) from about 1 ppm by weight, of a buffer; and

d) the balance carriers and adjunct ingredients.

This Application claims priority to U.S. Provisional Patent Application Ser. No. 60/176,090 filed Jan. 14, 2000.

### FIELD OF THE INVENTION

The present invention relates to compositions for controlling plant and flower moisture transpiration rates and thereby providing a means for extending the time in which plants and cut flowers can be utilized in aesthetic displays or floral arrangements.

### BACKGROUND OF THE INVENTION

Flowers have been inextricably linked to human culture since antiquity. Flowers have come to represent various aspects of life and to represent various facets of the human condition. As symbols of our society they speak directly. Flowers are never out of place regardless of the circumstances, inter alia, births, funerals, weddings, memorials.

Humans have cultivated and propagated flowers solely for their aesthetic value since most flowers are inedible. Incunabula describe various techniques for cutting and preserving flowers, inter alia, oriental flower varnishing, dipping blossoms into waxes or wax-like solutions. Contemporary practices include fashioning artificial flowers and blossoms from synthetic material, most notably polymers. However, all of these methods for preserving flowers, or attempts at flower imitation, fails to reproduce or replace the freshness of newly cut flowers.

The prior art has attempted to provide methods of preserving cut flowers in a fresh state, but the means are inadequate to provide flowers in a nearly original state for an enhanced period of time, for example, two to five times the expected period of use.

There is, therefore, a long felt need to provide the consumer or the grower of flowers which are to be cut and displayed for aesthetic purposes, with a system with significantly extends the duration in which the cut flowers maintain their original appearance.

### SUMMARY OF THE INVENTION

The present invention meets the aforementioned needs in that it has been surprisingly discovered that cut flowers can be preserved in a nearly original state for an extended period of time, in fact, in some instances a period which eclipses their aesthetic utility. It has been surprisingly discovered that by providing the cut flowers or plants with a suitable source of energy and moisture while effectively abating the restriction to nutrient uptake caused by microbial growth and ion concentration gradients, flowers can be cut and displayed without the pejorative effects of natural demise (senescence), inter alia, wilting (epinasty) or loss of petals, browning or discoloration of flower parts. The abatement of nutrient materials can be suitably established by controlling the type of nutrient and the type of antimicrobial employed.

Flowers are ubiquitous in that they can adapt to environmental or ecological stresses. For example, during times of drought or other circumstances of water deprivation, flowers regulate their growth to attenuate the effects which this moisture deprivation stress might have on their viability. This ability to self regulate their growth cycle ameliorates many of the pejorative consequences of water deprivation on flower survival. Once flowers are cut during harvesting, the natural regulatory systems, inter alia, respiration, water regulation, are abated. It has been surprisingly discovered that an artificial level of viability can be maintained by a system which controls the plant water intake/evaporation cycle. Although insufficient to induce or sustain reproductive viability, i.e. the production of pollen, seeds, etc., this system, nevertheless, maintains cut flowers in their natural condition for extended periods of time without the induction of discoloration, wilting, and petal loss.

The first aspect of the present invention relates to a composition for controlling plant and flower moisture transpiration, said composition comprising:

a) from about 0.1% by weight, of a source of energy;

b) from about 5 ppm by weight, of one or more antimicrobials;

c) from about 10 ppm by weight, of a buffer; and

d) the balance carriers and adjunct ingredients.

A second aspect of the present invention relates to a composition for controlling plant and flower moisture transpiration, said composition comprising:

a) from about 0.1% by weight, of a source of energy;

b) from about 5 ppm by weight, of one or more antimicrobials, at least one of said antimicrobials is an isothiazolone;

c) from about 10 ppm by weight, of a buffer; and

d) the balance carriers and adjunct ingredients.

Another aspect of the present invention relates to a composition for controlling plant and flower moisture transpiration, said composition comprising:

a) from about 0.1% by weight, of a source of energy;

b) from about 5 ppm by weight, of one or more antimicrobials, at least one of said antimicrobials having the formula:

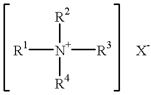

wherein R¹ and R² are each independently C₈-C₂₀ linear or branched alkyl, benzyl, and mixtures thereof; R³ and R⁴ are each independently C₁-C₄ alkyl, and mixtures thereof; X is an anion of sufficient charge to provide electronic neutrality;

c) from about 10 ppm by weight, of a buffer; and

b) the balance carriers and adjunct ingredients.

A further aspect of the present invention relates to a composition for controlling plant and flower moisture transpiration, said composition comprising:

a) from about 0.1% by weight, of a source of energy;

b) from 1 ppm to 200 ppm by weight, of an antimicrobial system; said system comprising”

i) from 1% to 99% by weight, of said system, of one or more isothiazolone antimicrobials;

ii) from 1% to 99% by weight, of said system, of one or more antimicrobials having the formula:

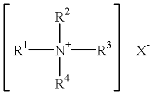

wherein R¹ and R² are each independently C₈-C₂₀ linear or branched alkyl, benzyl, and mixtures thereof; R³ and R⁴ are each independently C₁-C₄ alkyl, and mixtures thereof; X is an anion of sufficient charge to provide electronic neutrality;

c) from about 10 ppm by weight, of a buffer; and

b) the balance carriers and adjunct ingredients.

The present invention also relates to methods for extending the vase-life of a cut flower or plant.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference.

### DETAILED DESCRIPTION OF THE INVENTION

The present invention relates to aqueous compositions or granular compositions which can be added to water or other suitable carrier into which cut flowers are placed, said compositions are effective for controlling plant and flower moisture transpiration rates and thereby providing a means for extending the time in which plants and cut flowers can be utilized in aesthetic displays or floral arrangements. The present invention is achieved by controlling the intake of water and nutrients by a cut flower. The compositions of the present invention provide a source of energy and moisture to the cut flower while controlling the growth of microbes and, preferably, the calcium ion flux produced by the plant.

Without wishing to be limited by theory it has been found that control of the evaporation of water from cut flowers contributes to the enhanced duration in which flowers appear in their pre-harvested state. It has also been surprisingly discovered that certain anti-microbial compounds or anti-microbial systems will abate the growth of microbes which serve to diminish the viability of cut flowers. Without wishing to be limited by theory, it has been surprisingly discovered that certain prior art antimicrobials, inter alia, 8-hydroxy quinoline citrate actually enhance the growth of microorganisms after an initial diminution of their level. The pejorative consequences of microorganism growth in the display water (i.e. vase water) is not solely related to the aesthetics of the solution into which the flowers are placed, for example, milky appearance, formation of sediments, but instead the microorganisms occlude the xylem of the flower stem thereby attenuating the uptake of water and nutrients.

It has also been surprisingly discovered that once a suitable nutrient uptake has been established in the cut flower or plant, they begin to establish an equilibrium concentration of calcium ions between the plant cell and the interstitial water. It is preferred that this equilibrium be regulated. The proper equilibrium can be maintained by the presence of a water clarification agent. Therefore, depending upon the composition of the source water used to make up the solution of the second component, the amount of calcium sequestration will vary as a preferred adjunct ingredient of the composition.

For the purposes of the present invention the term “aesthetic utility” is defined herein as “the duration in which a flower retains its aesthetic appeal”. The end of aesthetic appeal may differ between species of plant or flower, however, non-limiting examples of a property which may contribute wholly or severally to a loss of aesthetic appeal include browning of petals, loss of petals, drooping or down turn of blossom, wilting, and shrinkage of plant mass together with collapse of plant tissue. In some instances, one manifestation of senescence may abate the usefulness of the flower, for example, the “browning” of petals may preclude the further use of a flower regardless of the lack of other conditions which tend to detract from the aesthetic quality of the cut flower.

The granular compositions of the present invention are added to water to make up a solution into which is placed the stem of a plant or flower. The water to which the composition is added can be household water, i.e. tap water, preferably said water comprises less than 3 grains of calcium, more preferably said water is distilled water, most preferably distilled water which is treated to remove any exogenic microorganisms. Or alternatively, the compositions of the present invention can be provided as a pre-formed solution, or as a concentrate which is further diluted prior to use by the formulator or consumer.

### Source of Energy

The compositions of the present invention comprise a source of energy or nutrients for sustaining the viability cut plants or flowers during the display period. The compositions of the present invention, prior to said compositions being dissolved in water or other suitable carrier or mixtures of carriers and water, to form a solution, comprise from about 75% by weight, of a source of energy. Other embodiments of the present invention comprise from about 90% by weight, of a source of energy while yet another embodiment comprises from about 99% by weight, of a source of energy. The compositions described herein may also comprise up to about 99.95% by weight, of an energy source. Suitable sources of energy include saccharide, oligosaccharide, polysaccharide, etc., and mixtures thereof regardless of form, provided the source of energy has sufficient water solubility. For the purposes of the present invention the term “sugar” or “sugars” will stand equally well for saccharide, oligosaccharide, polysaccharide, and “reducing sugars, non-reducing sugars and the like”. Non-limiting examples of sugars, which are a source of energy, suitable for any number of embodiments of the present invention, include aldopentoses such as ribose, arabinose, and xylose; aldohexoses such as allose, altrose, glucose, mannose, gulose, idose, galactose, and talose; ketohexoses such as fructose; monosaccharide derivatives such as alkyl-α-, alkyl-β-, aryl-α-, aryl-β-glycosides such as methyl-α-D-glucopyranoside and phenyl-α-D-glucopyranoside, and salicin; disaccharides such as lactose, maltose, cellobiose, gentiobiose, turanose, isomaltose, laminaribose, melibiose, sucrose, and trehalose; and trisaccharides such as raffinose and gentianose. Examples of easily available and inexpensive sourcse of energy include glucose and sucrose. Glucose is utilized by many of the embodiments described herein as a non-limiting example of a source of energy.

When complex sugars are taken into the plant, they are split into their constituent units, for example, sucrose into glucose and fructose, which results in a concentration gradient which further enhances the uptake of moisture.

### Antimicrobial

When microbes are allowed to grow in the vase water into which cut plants or flowers are placed, the microbes will occlude the xylem of the plant stem and abate the uptake of moisture and nutrients into the flower or plant leaves and petals. The present invention comprises one or more anti-microbial compounds which serve to abate the obstruction of fluid and source of energy uptake into the cut flower or plant. The compositions of the present invention comprise an “effective amount” of an anti-microbial or anti-microbial system. An anti-microbial system is defined herein as two or more anti-microbial compounds. The term “effective amount” is defined herein as the amount of an anti-microbial or anti-microbial system sufficient to abate the growth of microbes which act to occlude the uptake of nutrients by the cut flower or plant.

Embodiments of the present invention include final aqueous solutions comprising from about 1 ppm (0.0001%) with upper limits of from 100 ppm (0.01%) to 200 ppm (0.02%) by weight. Indeed, other embodiments comprise from about 5 ppm (0.0005%) to to ranges of about 50 ppm (0.005%) to about 100 ppm (0.01%) by weight, of an antimicrobial. When expressed as non-aqueous, granular compositions, or compositions prior to dissolving into a liquid carrier, comprise from about 0.1% on a dry weight basis of one or more anti-microbial compounds. Another embodiment comprisins from about 1% on a dry weight basis, of one or more anti-microbial compounds.

Embodiments of the present invention include granular compositions comprising from about 100 ppm (0.01%) with upper limits of from 10,000 ppm (1%) to 20,000 ppm (2%) by weight. Indeed, other embodiments comprise from about 500 ppm (0.05%) to to ranges of about 5000 ppm (0.5%) to about 10,000 ppm (1%) by weight, of an antimicrobial. When expressed as non-aqueous, granular compositions, or compositions prior to dissolving into a liquid carrier, comprise from about 0.01% on a dry weight basis of one or more anti-microbial compounds. Another embodiment comprisess from about 0.05% to about 0.1% on a dry weight basis, of one or more anti-microbial compounds.

The compositions of the present invention may also comprise antimicrobial systems which are a combination of two or more antimicrobials. Said systems will afford the formulator with the ability to target certain species of microorganisms which are characteristic of a specific plant species.

As a non-limiting example, an final aqueous solution which comprises 1% by weight, of a source of energy and 250 ppm (0.025%) of one of more anti-microbial compounds is formed from a dry (granular) composition comprising:

i) about 97.6% by weight, of a source of energy; and

ii) about 2.4% by weight, of an antimicrobial system.

One type of anti-microbial compounds are quaternary ammonium compounds having the formula:

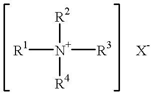

wherein R¹ and R² are each independently C₈-C₂₀ linear or branched alkyl, benzyl, and mixtures thereof, preferably R¹ and R² are each C₁₂ alkyl; or alternatively one of R¹ and R² is a mixture of n-alkyl units, inter alia, C₁₂, C₁₄, and C₁₆, and on of R¹ and R² is benzyl; R³ and R⁴ are each independently C₁-C₄ alkyl, and mixtures thereof, preferably R³ and R⁴ are each methyl; X is an anion of sufficient charge to provide electronic neutrality, preferably halogen, more preferably chlorine. Non-limiting examples of preferred antimicrobial is didodecyl dimethylammonium chloride and the admixture of C₁₂, C₁₄, and C₁₆ n-alkyl, benzyl dimethyl ammonium chlorides ex Lonza.

Another type of antimicrobial includes isothiazolones having the formula:

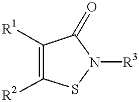

wherein R¹ and R² are each independently hydrogen, alkyl, alkenyl, halogen, cyano, and mixtures thereof or R¹ and R² can be taken together to form an aromatic or non-aromatic, heterocyclic or non-heterocyclic ring. R³ is hydrogen, alkyl, and mixtures thereof. A preferred R³ is methyl.

Non limiting examples of suitable isothiazolones include:

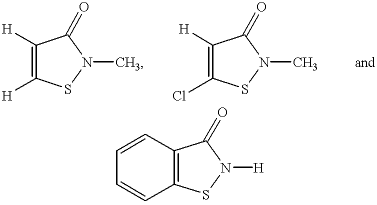

which can be combined, as in the case of Kathon® CG/ICP II ex Rohm and Haas (added embodiment) which is a combination of 2-methylisothizaol-3-one and 2-methyl-5-chloroisothizol-3-one. Another preferred anti-microbial, 1,2-benzisothiazolin-3-one, is sold under the name Proxel® GXL ex Zeneca. Anti-microbial of this class can be used at a level of from about 0.1 ppm (0.00001%), in other embodiments from about 1 ppm (0.0001%). The upper range of antimicrobials can be up to about 20 ppm (0.002%), yet other embodiments may limit this upper range to about 10 ppm (0.001%) by weight, of the final aqueous solution which serves as the vase solution.

### Buffers and Buffer Systems

The compositions of the present invention when used, have an acidic pH. What is meant herein by acidic pH is a pH which is lower than 7, or which has some amount of hydrogen ion present. Particularly useful embodiments have a pH of between 2 and about 5. Some embodiments are more narrow in range, that is from about about 3 to about 4 or from about 2 to about 3.5. The final pH range will be predicated on several factors including the selection of buffers or buffer systems, the type of embodiment and the scope of the formulators composition.

The aqueous compositions of the present invention comprise in one embodiment from about 0.0001% (1 ppm) by weight, of said buffer. Other embodiments comprise from 0.001% (10 ppm) to about 0.1% (1000 ppm) by weight, of said buffer. A particular embodiment comprises from about 0.01% (100 ppm) to about 0.016% (160 ppm) by weight, of a buffer system.

For dry granular compositions which are to be dissolved in a suitable carrier, on embodiment comprises from about 0.98% by weight, of a buffer. Other embodiments comprise from 2% to about 10% by weight, of a buffer. A particular embodiment comprises from 1.5 to 2% by weight, of said buffer.

Organic acid buffers and buffering systems may be used by the formulator as well as buffers and buffering systems which derive from inorganic acids. For example, citric acid may be used directly as a buffer, or in another embodiment, a citric acid/sodium citrate admixture may be used to create specific system. Sodium hydrogen phosphate/disodium hydrogen phosphate buffer systems are also suitable for the present invention.

Non-limiting examples of sutiable acids include those selected from the group consisting of citric acid, itaconic acid, malonic acid, maleic acid, caffeic acid, succinic acid, adipic acid, sebacic acid, and salts thereof. Of course, the free acid and salts may be added as admixtures and admixtures of any acids and acid salts can be employed.

### Adjunct Ingredients

The compositions of the present invention can optionally comprise one or more adjunct ingredients. A preferred adjunct ingredient according to the present invention is a calcium chelant or calcium sequestrant. Non-limiting examples of calcium sequestrants include sodium tripolyphosphate, finely divided zeolite including zeolite A, zeolite X, and zeolite Y, ethylenediamine, and mixtures thereof. A further example of a preferred adjunct ingredient is selected from the group consisting of surfactants, fragrance raw materials, pro-fragrances, pro-accords, dye, colorants, and mixtures thereof. Suitable pro-fragrances and pro-accords are described in U.S. Pat. No. 5,919,752 Morelli et al., issued Jul. 6, 1999; U.S. Pat. No. 5,756,827 Sivik, issued May 26, 1998; U.S. Pat. No. 5,744,435 Hartman et al., issued Apr. 25, 1998; and U.S. Pat. No. 5,965,767 Sivik et al., issued Oct. 12, 1999 all of which are incorporated herein by reference.

In one aspect of the present invention, the compositions are prepared as dry, powdered mixtures which are stored and shipped as such and dissolved in water immediately prior to use as cut flower preservative solutions. When in the form of dry powders, the formulations of this invention are packaged in bulk for end use, as in containers having a tightly-fitting lid such as screw-capped or snap-capped bottles or, preferably are packaged in plastic or foil packets containing the required amount of material for a single use.

A dry composition comprising 99.5% by weight, glucose and the balance an anti-microbial, when 1 gm of said dry composition is dissolved in 1 liter of distilled water will provide approximately 0.1% by weight, of a source of energy and approximately 5 ppm of said anti-microbial. The formulations of the compositions, depending upon the relative levels of components, are dissolved in water just prior to use at a concentration ranging from about to about 20 g/liter. Other embodiments can range from 1 g/liter to about 15 g/liter. Yet other embodiments range from about 5 g/liter or from about 7 g/liter to about 10 g/liter. For a typical arrangement of cut flowers, the volume of water in a vase is about one-half to one liter. Therefore, a preferred package of the second component of the present invention is a foil or plastic packet containing about 2.5 grams to 3 grams of material.

An example of a granular composition which is diluted with water or a mixture of water and other carriers comprises:

a) 1000 ppm (0.1%) a source of energy;

b) 5 ppm (0.0005%) an antimicrobial;

c) 1 ppm (0.0001%) a buffer; and

d) the balance a carrier.

Prior to dissolving the compostion in solution, the granular product comprises:

a) 99.94% by weight, a source of energy;

b) 0.05% by weight, an antimicrobial; and

c) 0.01% by weight, a buffer.

In another embodiment of the present invention, the source of water can be critical. For example, certain regions have native water, household or otherwise, which contains high levels of calcium. Therefore the compositions may be delivered as a concentrate in de-ionized, distilled water which when added to a native water supply provides a solution having a sufficiently low level of calcium to establish post-harvest plant viability. Alternatively, the compositions can be delivered as a final solution in non-calcium containing water.

The following are non-liming examples of the compositions which comprise the present invention.

TABLE I

| | weight % | | | |
|---|---|---|---|---|
| Ingredients | 1 | 2 | 3 | 4 |
| Source of energy¹ | 1.0 | 1.0 | — | — |
| Source of energy² | — | — | 1.5 | — |
| Source of energy³ | — | — | — | 1.25 |
| Antimicrobial⁴ | 0.01 | 0.01 | 0.025 | 0.025 |
| Antimicrobial⁵ | 0.01 | 0.01 | — | — |
| Antimicrobial⁶ | 0.005 | 0.005 | — | — |
| Calcium sequestrant⁷ | | 1.0 | — | 1.0 |
| Carrier⁸ | balance | balance | balance | balance |

¹Sucrose.
²Glucose.
³Isomaltose.
⁴Didodecyl dimethylammonium chloride.
⁵Admixture of C₁₂, C₁₄, and C₁₆ n-alkyl, benzyl dimethyl ammonium chlorides ex Lonza.
⁶1,2-Benzisothiazolin-3-one sold under the name Proxel ® GXL ex Zeneca.
⁷Sodium tripolyphosphate.
⁸Distilled water.

TABLE II

| | weight % | | | |
|---|---|---|---|---|
| Ingredients | 5 | 6 | 7 | 8 |
| Source of energy¹ | 1.0 | 1.0 | — | — |
| Source of energy² | — | — | 1.5 | — |
| Source of energy³ | — | — | — | 1.25 |
| Antimicrobial⁴ | 0.001 | 0.001 | 0.001 | 0.001 |
| Calcium sequestrant⁵ | | 1.0 | — | 1.0 |
| Carrier⁶ | balance | balance | balance | balance |

¹Sucrose.
²Glucose.
³Isomaltose.
⁴Kathon ICP/CG II (Rohm & Haas).
⁵Sodium tripolyphosphate.
⁶Distilled water.

TABLE III

weight %

| Ingredients | 9 | 10 | 11 | 12 |
|---|---|---|---|---|
| Source of energy¹ | 0.75 | 1.0 | 1.5 | 2.0 |
| Antimicrobial² | 0.001 | 0.001 | 0.001 | 0.001 |
| Antimicrobial³ | 0.004 | 0.004 | 0.004 | 0.004 |
| Citric acid | 0.01 | 0.01 | 0.01 | 0.01 |
| Sodium Citrate | 0.006 | 0.006 | 0.006 | 0.006 |
| Carrier⁴ | balance | balance | balance | balance |

¹Glucose.
²Kathon ® ICP/CG II (Rohm & Haas).
³Bartac ® 2250 (Lonza).
⁴Distilled, de-ionized water.

TABLE IV

weight %

| Ingredients | 13 | 14 | 15 | 16 |
|---|---|---|---|---|
| Source of energy¹ | 0.75 | 1.0 | 1.5 | 2.0 |
| Antimicrobial² | 0.001 | 0.001 | 0.001 | 0.001 |
| Antimicrobial³ | 0.004 | 0.004 | 0.004 | 0.004 |
| Citric acid | 0.01 | 0.01 | 0.01 | 0.01 |
| Sodium Citrate | 0.006 | 0.006 | 0.006 | 0.006 |
| Carrier⁴ | balance | balance | balance | balance |

¹Glucose.
²Niolone ® M-50 (Rohm & Haas).
³Bartac ® 2250 (Lonza).
⁴Distilled, de-ionized water.

TABLE V

weight %

| Ingredients | 17 | 18 | 19 | 20 |
|---|---|---|---|---|
| Source of energy¹ | 0.75 | 1.0 | 1.5 | 2.0 |
| Antimicrobial² | 0.001 | 0.001 | 0.001 | 0.001 |
| Antimicrobial³ | 0.004 | 0.004 | — | — |
| Antimicrobial⁴ | — | — | 0.004 | 0.004 |
| Citric acid | 0.01 | 0.01 | 0.01 | 0.01 |
| Sodium Citrate | 0.006 | 0.006 | 0.006 | 0.006 |
| Carrier⁵ | balance | balance | balance | balance |

¹Glucose.
²Kathon ® ICP/CG II (Rohm & Haas).
³Bartac ® 2050 (Lonza).
⁴Bartac ® LF-80 (Lonza).
⁵Distilled, de-ionized water.

What is claimed is:

1. A composition for controlling plant and flower moisture transpiration, said composition comprising:
a) from about 0.5% by weight, of a source of energy;
b) from about 0.05 % by weight, of one or more antimicrobials; and
c) the balance carriers and adjunct ingredients.

2. An aqueous composition for controlling plant and flower moisture transpiration, said composition comprising:
a) from about 0.1% by weight, of a source of energy;
b) from about 5 ppm by weight, of one or more antimicrobials;
c) from about 1 ppm by weight, of a buffer; and
d) the balance carriers and adjunct ingredients.

3. A composition according to claim 2 wherein said source of energy comprises a saccharide, oligosaccharide, polysaccharide, or mixtures thereof.

4. A composition according to claim 3 wherein said source of energy comprises sucrose, glucose, or mixtures thereof.

5. A composition according to claim 4 wherein said source of energy is glucose.

6. A composition according to claim 3 wherein said source of energy is an oligosaccharide.

7. A composition according to claim 1 comprising from about 0.5% to about 10% by weight, of a source of energy.

8. A composition according to claim 7 comprising from about 1% by weight, of a source of energy.

9. A composition according to claim 8 comprising to about 5% by weight, of a source of energy.

10. A composition according to claim 1 wherein said antimicrobial is selected from the group consisting of 2-methyl-4-isothiazolin-3-one, 5-chloro-2-methyl-4-isothiazolin-3-one, and mixtures thereof.

11. A composition according to claim 1 wherein said antimicrobial has the formula: 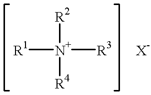
wherein R¹ and R² are each independently C₈-C₂₀ linear or branched alkyl, benzyl, and mixtures thereof; R³ and R⁴ are each independently C₁-C₄ alkyl, and mixtures thereof; X is an anion of sufficient charge to provide electronic neutrality.

12. A composition according to claim 2 wherein said buffer is selected from the group consisting of citric acid, itaconic acid, malonic acid, maleic acid, caffeic acid, succinic acid, adipic acid, sebacic acid, and salts thereof.

13. A composition according to claim 12 wherein said buffer comprises citric acid and sodium citrate.

14. A composition according to claim 13 comprising from 10 ppm to about 1000 ppm, citric acid and sodium citrate wherein the ratio of acid to sodium salt is from 20:3.

15. A composition according to claim 14 comprising 0.016% by weight of a citric acid/sodium citrate buffer system wherein the ratio of acid to sodium salt is 5:2.

16. A composition according to claim 2 having a pH of from about 2 to about 5.

17. A composition according to claim 16 wherein said pH is from about 3 to about 4.

18. A composition according to claim 17 having a pH of about 4.

19. An aqueous composition for controlling plant and flower moisture transpiration, said composition comprising:
a) from about 0.1% by weight, of a source of energy;
b) from about 1 ppm by weight, of an antimicrobial system, said system comprising:
i) from 1% to 99% by weight, of said system, of one or more isothiazolone antimicrobials;
ii) from 1% to 99% by weight, of said system, of one or more antimicrobials having the formula: 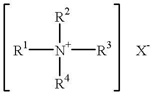
wherein R¹ and R² are each independently C₈-C₂₀ linear or branched alkyl, benzyl, and mixtures thereof; R³ and R⁴ are each independently C₁-C₄ alkyl, and mixtures thereof; X is an anion of sufficient charge to provide electronic neutrality;
c) from about 10 ppm by weight, of a buffer; and
d) the balance carriers and adjunct ingredients.

20. A composition according to claim 19 wherein at least one antimicrobial comprises R¹ and R² are each C₁₂ alkyl; R³ and R⁴ are each methyl; X is chlorine.

21. A composition according to claim 19 wherein at least one antimicrobial comprises R¹ is a mixture of C₁₂-C₁₆ alkyl; R² is benzyl, R³ and R⁴ are each methyl; X is chlorine.

22. A composition according to claim 19 wherein said antimicrobial is an antimicrobial system comprising:
i) from 10 ppm to 200 ppm by weight, of didecyl dimethyl ammonium chloride;
ii) from 10 ppm to 200 ppm by weight, of n-alkyl dimethyl benzyl ammonium chloride wherein n-alkyl comprises an admixture of C₁₂, C₁₄, and C₁₆ linear alkyl chains; and
iii) from 1 ppm to 100 ppm by weight, of 1,2-benzisothiazolin-3-one.

23. A composition according to claim 22 wherein said antimicrobial is an antimicrobial system comprising:
i) 100 ppm by weight of said composition, of didecyl dimethyl ammonium chloride;
ii) 100 ppm by weight of said composition, of n-alkyl dimethyl benzyl ammonium chloride wherein n-alkyl comprises an admixture of C₁₂, C₁₄, and C₁₆ linear alkyl chains; and
iii) 50 ppm by weight of said composition, of 1,2-benzisothiazolin-3-one.

24. A system according to claim 19 further comprising a calcium ion sequestrant.

25. A composition according to claim 19 having a pH of from about 2 to about 5.

26. A composition according to claim 25 wherein said pH is from about 3 to about 4.

27. A composition according to claim 26 having a pH of about 4.

28. A composition according to claim 19 wherein said source of energy comprises a saccharide, oligosaccharide, polysaccharide, or mixtures thereof.

29. A composition according to claim 28 wherein said source of energy comprises sucrose, glucose, or mixtures thereof.

30. A composition according to claim 29 wherein said source of energy is glucose.

31. A non-liquid composition for controlling plant and flower moisture transpiration, said composition comprising:
a) from about 75% by weight, of a source of energy;
b) from about 0.05% by weight, of one or more antimicrobials;
c) from about 0.01% by weight, of a buffer; and
d) the adjunct ingredients.

32. A composition according to claim 31 comprising from about 90% by weight, of a source of energy.

33. A composition according to claim 32 comprising from about 95% by weight, of a source of energy.

34. A composition according to claim 33 comprising from about 99% by weight, of a source of energy.

35. A composition according to claim 31 comprising from about 0.01% to about 2% by weight, of an anti-microbial.

36. A composition according to claim 35 comprising from about 0.05% to about 0.5% by weight, of an anti-microbial.

37. A composition according to claim 36 comprising from about 0.05% to about 0.1% by weight, of an anti-microbial.

38. A granular composition for dilution by a carrier, said composition for controlling plant and flower moisture transpiration, said composition comprising:
a) from about 75% by weight, of a source of energy;
b) from about 0.01% by weight, of an antimicrobial system, said system comprising:
i) from 1% to 99% by weight, of said system, of one or more isothiazolone antimicrobials;
ii) from 1% to 99% by weight, of said system, of one or more antimicrobials having the formula: 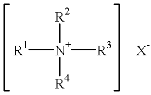
wherein R¹ and R² are each independently C₈-C₂₀ linear or branched alkyl, benzyl, and mixtures thereof; R³ and R⁴ are each independently C₁-C₄ alkyl, and mixtures thereof; X is an anion of sufficient charge to provide electronic neutrality;
c) from about 0.98% by weight, of a buffer; and
d) the balance adjunct ingredients.

39. A composition according to claim 38 wherein said buffer is selected from the group consisting of citric acid, itaconic acid, malonic acid, maleic acid, caffeic acid, succinic acid, adipic acid, sebacic acid, and salts thereof.

40. A composition according to claim 39 wherein said buffer comprises citric acid and sodium citrate.

41. A method for enhancing the longevity of cut flowers comprising the step of contacting the cut ends of said cut flowers with a vase additive solution, said solution comprising:
a) from about 75% by weight, of a source of energy;
b) from about 0.01% by weight, of an antimicrobial system, said system comprising:
i) from 1% to 99% by weight, of said system, of one or more isothiazolone antimicrobials;
ii) from 1% to 99% by weight, of said system, of one or more antimicrobials having the formula: 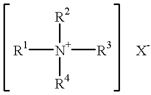
wherein R¹ and R² are each independently C₈-C₂₀ linear or branched alkyl, benzyl, and mixtures thereof; R³ and R⁴ are each independently C₁-C₄ alkyl, and mixtures thereof; X is an anion of sufficient charge to provide electronic neutrality;
c) from about 0.98% by weight, of a buffer; and
d) the balance adjunct ingredients.
